(19) United States
(12) Patent Application Publication
(10) Pub. No.: US 2016/0338836 A1
(43) Pub. Date: Nov. 24, 2016

(54) PRELIMINARILY BONDED MULTILAYER FILM FOR COVERING A BONE DEFECT SITE

(71) Applicant: Dietmar SONNLEITNER, Salzburg (AT)

(72) Inventor: Dietmar SONNLEITNER, Salzburg (AT)

(21) Appl. No.: 15/227,384

(22) Filed: Aug. 3, 2016

Related U.S. Application Data
(63) Continuation of application No. PCT/AT2015/000015, filed on Feb. 3, 2015.

(30) Foreign Application Priority Data
Feb. 5, 2014 (AT) 84/2014

(51) Int. Cl. A61F 2/28

(52) U.S. Cl. CPC A61F 2/2803; A61F 2/2846; A61F 2210/0004; A61F 2210/0076

(57) ABSTRACT

The invention relates to a preliminarily bonded multilayer film (1) for covering a bone defect site (2), in particular in the region of a jaw bone, the film (1) comprising at least one shaping molding layer (3) for molding the film (1) onto the bone defect site (2) and at least one cover layer (4, 4a, 4b) for covering the bone defect site (2), the molding layer (3) and the at least one cover layer (4, 4a, 4b) being substantially completely resorbable.

The invention concerns a pre-bonded multilayer film for covering a bone defect site, in particular in the region of a jawbone.

Known films or membranes for covering bone defect sites are used for example in the region of jawbones or for jaw augmentation to reconstruct a jawbone in the case of osteoporosis or bone loss which can occur in the extraction of a tooth or as a consequence of an inflammatory process around a natural tooth. Such films frequently involve a shaped structure of titanium which is arranged on a Teflon membrane and which is shaped over the bone defect site so that there is a cavity between the film and the bone defect site, in which bone material and in the case of natural teeth also the periodontal apparatus can regrow. Fixing of the film is usually effected with resorbable or metal pins or screws which are fixed through the film to the jawbone. Alternatively the film can also be fixed by adhesive to the support or the jawbone. With implants which are fitted at the same time the film can also be fixed to the implant head. As bone regeneration requires several months a second operation is necessary after bone reconstruction has been effected, with a Teflon membrane, in order to remove the film or Teflon membrane from the body again.

The object of the invention is to avoid the above-described disadvantages and to provide a pre-bonded multilayer film which is improved over the state of the art. In particular the invention aims to avoid a further operation for removal of the film.

According to the invention that object is attained by the features of claim 1. Advantageous configurations of the invention are recited in the appendant claims.

According to the invention it is therefore provided that the film includes a shaping molding layer for shaping the film to the bone defect site and at least one cover layer for covering the bone defect site, wherein the molding layer and the at least one cover layer are substantially completely resorbable.

Because therefore the film or membrane is overall completely resorbable in the body by being broken down in the body for example by hydrolysis the implementation of a further operation for removal of the film is no longer required. In other words in that case only one operation is required for fitting the film.

The film includes a shaping molding layer which serves for shaping molding of the film to the bone defect site and by which a cavity can be formed between the bone defect site and the film, so that bone growth can take place in that cavity. For advantageous bone regeneration the cavity can also contain bone substitute materials and/or carriers for drugs, growth factors and/or other substances which promote and protect healing and bone formation. The cavity is maintained by the cavity-forming and cavity-retaining molding layer until the cavity is filled up by further growth of bone material.

In addition the film includes at least one cover layer for covering the bone defect site. That cover layer which for example can be in the form of a membrane serves for covering over and sealing off the bone defect site to avoid soft tissue from penetrating into the bone defect site. In order further to improve fitting of the film and sealing off the bone defect site the at least one cover layer can also be of such a nature that it sticks to a gum surrounding the bone defect site.

The fact that the shaping molding layer and the at least one cover layer are provided in the form of a multilayer film which is already pre-bonded affords an easily handlable film for covering a bone defect site, which links the space-forming properties of the molding layer to the sealing properties of the cover layer and which in addition is substantially completely resorbed in the body. The individual layers of the proposed film (molding layer and at least one cover layer) can be mechanically and/or chemically connected together. It can therefore preferably be provided that the molding layer and the at least one cover layer are mechanically and/or chemically connected together.

In a preferred embodiment it can be provided that the molding layer and the at least one cover layer are substantially completely resorbable in different periods of time. Thus for example by virtue of the nature of the molding layer and the at least one cover layer it is possible to provide that the molding layer resorbs more quickly than the at least one cover layer. In general differing degrees of resorption capability in respect of the molding layer and the at least one cover layer afford great degrees of freedom in regard to the design of the film in relation to its resorption capability.

It can be provided that the film is substantially completely resorbable overall in a period of time of between about 3 and 12 months, preferably between about 4 and 6 months. That is the period of time with which bone reconstruction has occurred in a normal situation.

To permit good shaping molding to the bone defect site and stable cavity formation between the film and the bone defect site it can be provided that the molding layer is stiffer than the at least one cover layer. In that case the greater stiffness of the molding layer serves to form a cavity for bone construction and also to maintain that cavity for the period of time required for bone regeneration. Once again good coverage and sealing of the bone defect site can be achieved by a stiffness of the at least one cover layer, that is less in comparison with the molding layer.

Preferably it can be provided that the molding layer, possibly together with the at least one cover layer, is adapted to be mechanically and/or thermally and/or chemically deformable. Thus in particular the molding layer can be in the form of a layer which is substantially stable in respect of shape and which can be deformed under mechanical, thermal or chemical influence and which after that deformation regains a sufficient stability in respect of shape to maintain the cavity to be formed for bone growth, for the required period of time. The at least one cover layer can be flexible and preferably elastic to permit good coverage and sealing of the bone defect site.

Mechanical deformation can be effected in that case for example by bending with a forceps. That is a suitable method of shaping molding in particular for comparatively thin molding layers (for example in the region of between about 0.10 mm and about 0.5 mm). For thicker molding layers (for example thicker than about 0.5 mm) thermal deformation of a molding layer for shaping molding may be desirable. A corresponding thermal deformation procedure can be achieved in that case for example by means of a thermal bar having a hot tip or surface, by way of heated prefabricated models or in a hot water bath with a sterile saline solution.

For good resorption capability of the proposed film it can be provided that the at least one cover layer at least partially and preferably substantially completely comprises a bioresorbable collagen material. In that case it can be provided that the bioresorbable collagen material includes type-I-collagen and/or type-III-collagen. The collagen material can originate for example from bovine Achilles tendons.

For good resorption capacity on the part of the proposed film it can also be provided that the molding layer at least partially and preferably substantially completely comprises a bioresorbable polymer material. The bioresorbable polymer material can also involve a co-polymer material.

A particular variant provides that the bioresorbable polymer material includes lactic acid, preferably L-lactic acid, and/or derivatives thereof. It is advantageous in that respect if the proportion of lactic acid in the bioresorbable polymer material is at least 70%, preferably between about 80% and 95%, particularly preferably substantially about 82%.

In addition it can be provided that the bioresorbable polymer material includes glycolic acid. It is advantageous in that respect if the proportion of glycolic acid in the bioresorbable polymer material is at most 30%, preferably between about 15% and 20%, particularly preferably substantially about 18%. Depending on the respective composition of the molding layer it can be provided that the molding layer is substantially stable in respect of shape and nonetheless substantially completely resorbable.

In a further preferred embodiment it can be provided that the molding layer and the at least one cover layer have different surface areas. In that respect it can be provided that the molding layer involves a smaller surface area than the at least one cover layer. If the at least one cover layer covers over the molding layer by virtue of its lesser surface area it is possible to achieve particularly good coverage and thus also sealing of the bone defect site.

Preferably it can be provided that the at least one cover layer and/or the molding layer is or are substantially flat throughout. A film contour which is advantageous for shaping molding to the bone defect site can in that case be achieved for example by suitably cutting the film.

It is particularly desirable however if the molding layer has a shaping structure for shaping molding to the bone defect site. In that case it can be provided that the shaping structure has at least portion-wise a convexly and/or concavely curved edge and/or at least portion-wise a convexly and/or concavely curved shape. In other words the shaping structure can have for example areal—convexly and/or concavely curved—projections and can thus have a convexly and/or concavely curved edge. Alternatively or in addition the shaping structure as a whole can also be of a correspondingly convexly and/or concavely curved shape.

It is particularly advantageous if the shaping structure has at least one strut-shaped shaping molding element. The strut-shaped or lug-shaped shaping molding elements can in that case be shaped loop-like over the bone defect site and can permit any cavity shape.

A particularly advantageous embodiment of the invention is one in which the shaping structure is substantially grid-shaped. The grid-shaped structure in that case forms a reinforcing grid which permits the formation of the plurality of any desired cavity shapes.

It can also be provided that the shaping structure is provided by at least one reinforcement of the molding layer. In particular if the molding layer is applied in the form of a hardening liquid or a hardening gel to the at least one cover layer, it is desirable if the shaping structure can be achieved merely by applying more liquid or gel in the region of the shaping structure. In that case for example the molding layer can be of differing thicknesses.

A particular variant provides that the film has a carrier layer for at least one substance which is arranged or which is to be arranged thereon. The substances arranged or to be arranged on the carrier layer can involve drugs, growth factors and/or other substances which promote and protect healing and bone formation. The carrier layer can preferably be arranged at a side of the film, that is to be facing towards the bone defect site, and can at least partially and preferably substantially completely comprise a bioresorbable collagen material.

It can also be provided that corresponding substances are applied directly to the molding layer and/or the at least one cover layer. It can also be provided that the surface or side of the film, that is to be facing towards the bone defect site, itself serves as a carrier for the above-described substances by for example that side or surface of the film having appropriate roughness.

Depending on the respective situation of use the proposed film or membrane can also be provided in pre-cut and/or pre-shaped form. In that case for example a desired form of cut and/or a desired 3D deformation of the film can be effected in accordance with a data processing-aided planning procedure.

Further details and advantages of the present invention are described by means of the specific description hereinafter. In the drawing:

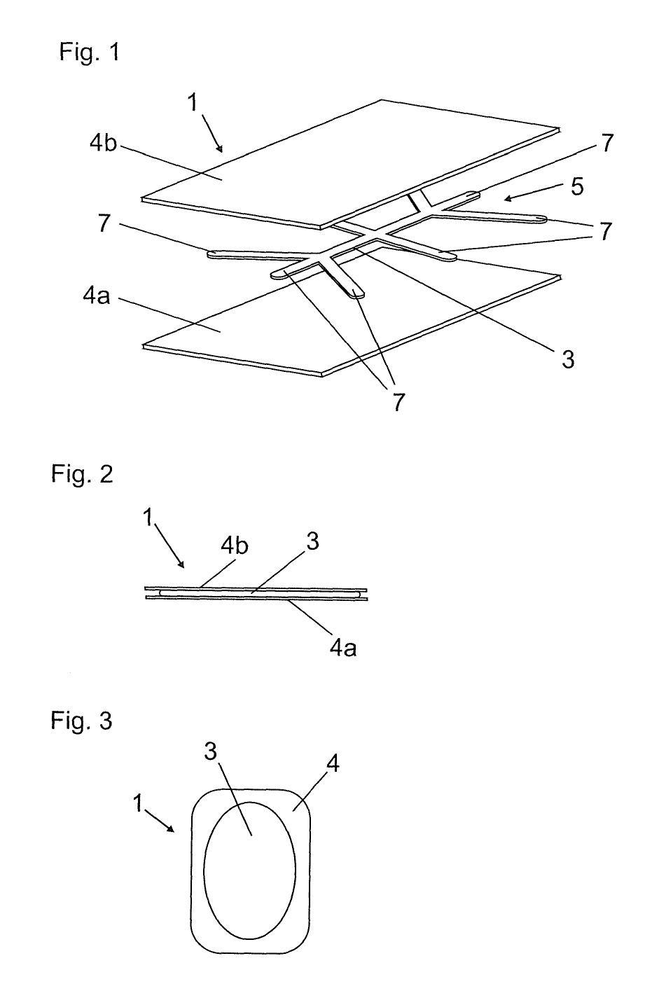

FIG. 1 shows a perspective exploded view of an embodiment of the proposed pre-bonded multilayer film,

FIG. 2 shows a side view of the proposed pre-bonded multilayer film shown in FIG. 1,

FIGS. 3-7 show plan views of various embodiments of the proposed pre-bonded multilayer film,

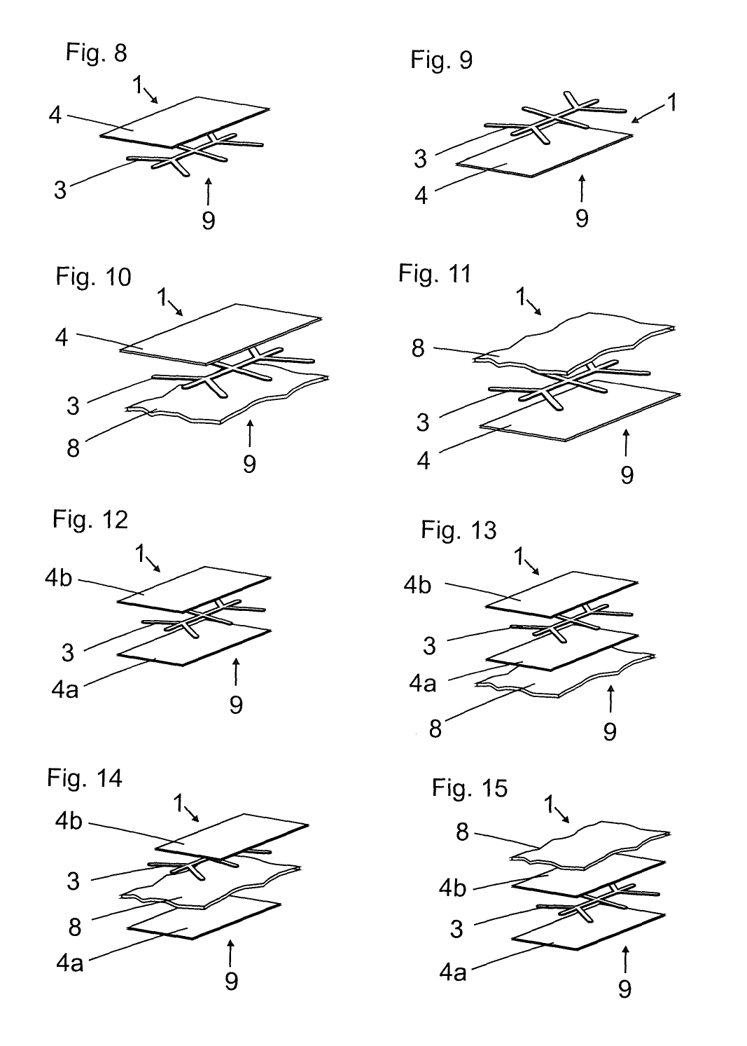

FIGS. 8-15 show perspective exploded views of a number of embodiments of the proposed pre-bonded multilayer film,

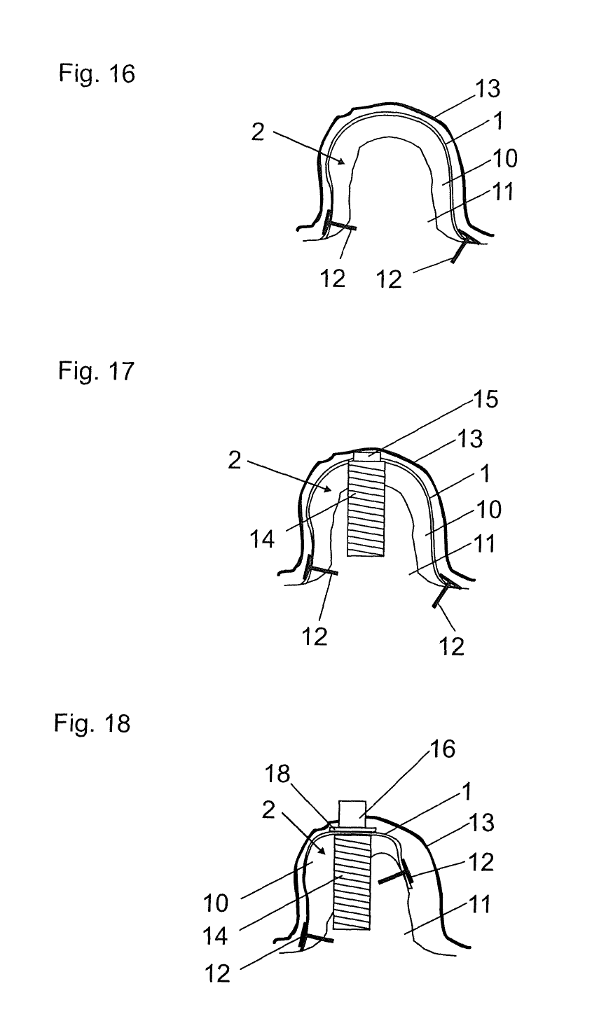

FIG. 16 shows a proposed pre-bonded multilayer film arranged at a bone defect site of a jawbone,

FIG. 17 shows a proposed pre-bonded multilayer film arranged at a bone defect site of a jawbone with implant,

FIG. 18 shows a proposed pre-bonded multilayer film arranged at a bone defect site of a jawbone with implant, support disk and post,

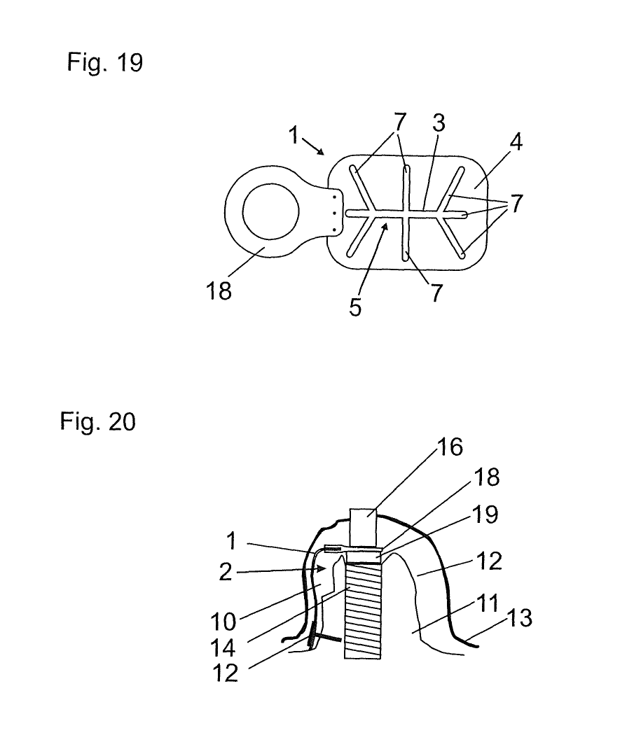

FIG. 19 shows a proposed pre-bonded multilayer film clamped in a groove in a support disk,

FIG. 20 shows a proposed pre-bonded multilayer film clamped in a groove in a support disk and arranged at a bone defect site of a jawbone with implant and post,

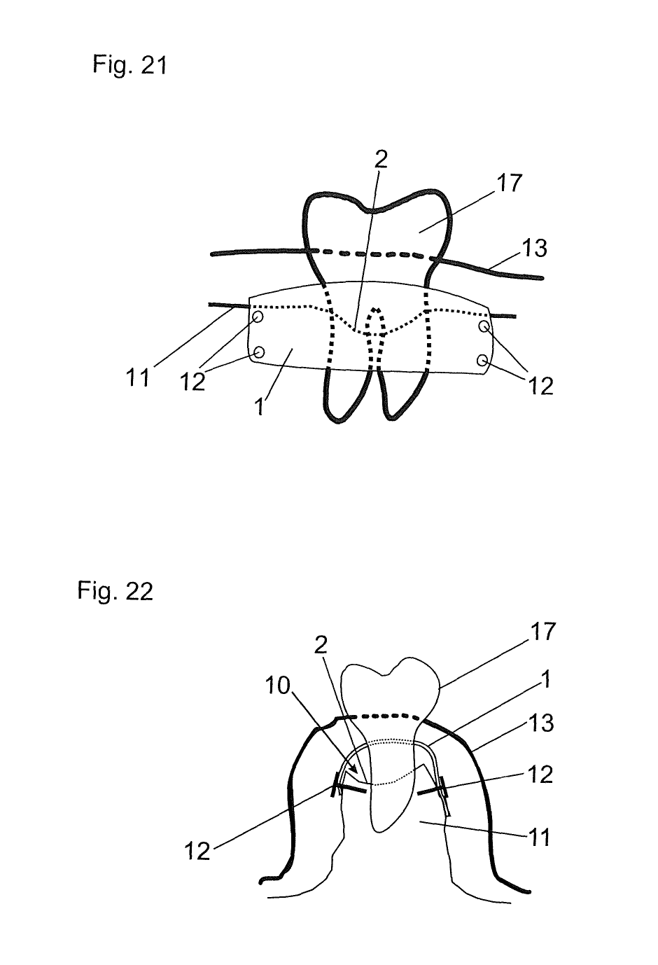

FIG. 21 shows a proposed pre-bonded multilayer film arranged at a bone defect site around a natural tooth, and

FIG. 22 shows a sectional view of a proposed pre-bonded multilayer film arranged at a bone defect site around a natural tooth.

FIG. 1 shows a perspective exploded view of a proposed pre-bonded multilayer film 1. The film 1 includes a molding layer 3 and two cover layers 4a and 4b. The molding layer 3 is stiffer than the cover layers 4a and 4b and has a shaping structure 5. The shaping structure 5 includes a plurality of strut-shaped shaping molding elements 7 which serve to shape the film 1 over a bone defect site 2 (not shown here), wherein the film 1 can be well shaped by the shaping molding elements 7 to a bone 11, which is still present, at the bone defect site 2 (see for example FIG. 16). The shaping structure 5 is overall of a substantially grid-shaped configuration and thus permits the provision of any surface shapes for the film 1 so that, in conjunction with a bone defect site 2, any desired cavity shapes can be formed between the film 1 and the bone defect site 2.

The molding layer 3 and the cover layers 4a and 4b respectively comprise a bioresorbable material so that the film 1 overall can be substantially completely resorbed in the body. By virtue of the provision of two cover layers 4a and 4b, between which the molding layer 3 is embedded, it is possible in particular to control the resorption speed and mechanical strength of the molding layer 3.

The cover layers 4a and 4b can for example comprise bioresorbable collagen membranes which on the one hand by virtue of their softness can well cover a bone defect site 2 and which on the other hand can be well secured by adhesive to a gum 13 surrounding the bone defect site 2 so as to ensure that the bone defect site 2 is well sealed off.

The molding layer 3 can for example comprise a bioresorbable polymer material or co-polymer material. In particular the molding layer 3 can include for example about 82% L-lactic acid and about 18% glycolic acid. Such a choice of material affords a molding layer 3 which is substantially stable in shape and which can be adapted to be mechanically, thermally and/or chemically deformable for shaping molding to a bone defect site 2, wherein the molding layer 3, after such deformation, is again substantially stable in respect of shape. By virtue of the stiffness and stability of shape of the molding layer 3 it is thus possible to provide a cavity for bone regeneration between the film 1 and a bone defect site 2 and to also maintain it for the period of bone regeneration.

FIG. 2 shows a side view of the pre-bonded multilayer film 1 shown in FIG. 1.

FIG. 3 shows a plan view of a further variant of the proposed film 1 which in this embodiment is of a double-layer nature and includes a molding layer 3 and a cover layer 4. Both the molding layer 3 and also the cover layer 4 are substantially flat. The film 1 can be cut to shape as desired to permit good molding to a bone defect site 2 depending on the respective situation of use.

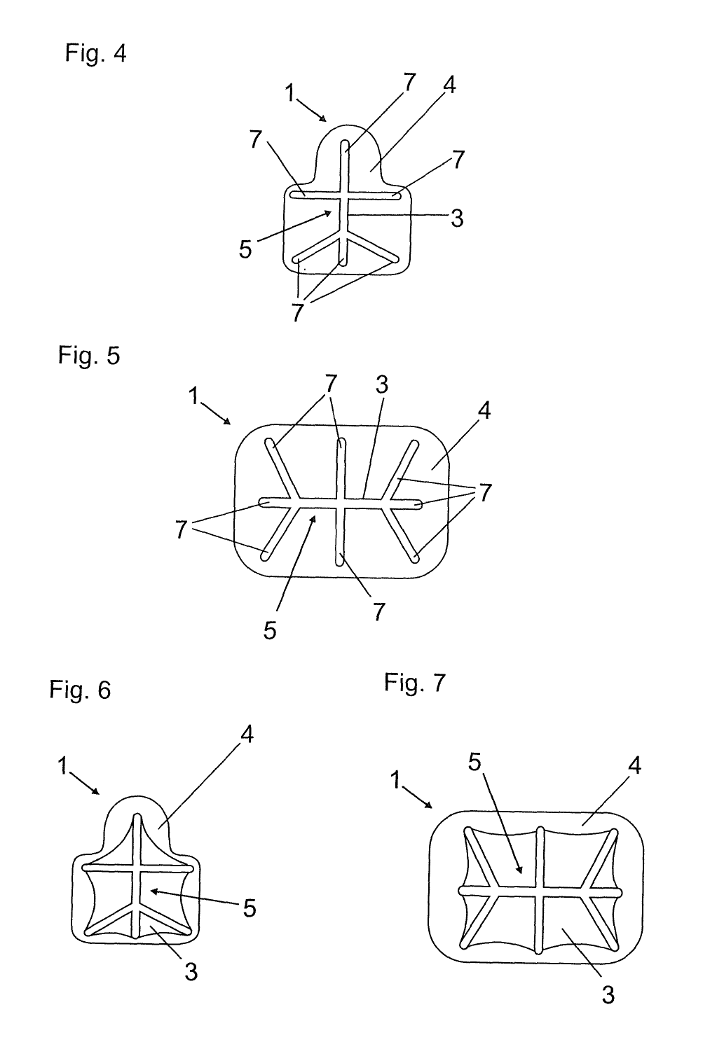

FIGS. 4 and 5 show two further embodiments of a proposed double-layer film 1 with different external contours for the cover layer 4 and different shaping structures 5 for the molding layer 3.

FIGS. 6 and 7 show further examples of proposed films 1, wherein the molding layer 3 in the examples shown here was applied to the cover layer 4 in the form of a gel and subsequently hardened. The molding layers 3 shown here each include a shaping structure 5 which was achieved for example by more gel being applied in the regions of the shaping structure 5 so that the molding layers 3 involve different layer thicknesses. In the region of a shaping structure 5 a molding layer 3 is of a respectively larger layer thickness than in the other regions of the molding layer 3.

FIGS. 8 through 15 respectively show perspective exploded views of further embodiments of a proposed film 1. The side 9 of a film 1, which faces downwardly in each of the Figures, is the side 9 of the film 1, that is to face towards a bone defect site 2.

The examples in FIGS. 8 and 9 are of a double-layer structure and each include a molding layer 3 and a cover layer 4, wherein the molding layer 3 occupies a smaller surface area than the cover layer 4. The examples in FIGS. 10 and 11 are of a triple-layer structure and, besides a molding layer 3 and a cover layer 4, each include a carrier layer 8 on which can be applied substances like for example drugs, growth factors and/or other substances for promoting and protecting healing and bone formation.

The examples in FIGS. 12 through 15 each have a molding layer 3 and two cover layers 4a and 4b, wherein the molding layer 3 occupies a smaller surface area than the cover layers 4a and 4b. The examples in FIGS. 13 through 15 each additionally have a carrier layer 8 which can be equipped with corresponding substances (as described above in relation to FIGS. 10 and 11).

FIG. 16 shows a sectional view of a jawbone 11 with a bone defect site 2. To permit bone construction at the bone defect site 2 a proposed pre-bonded multilayer film 1 is suitably moldingly shaped over the bone defect site 2 and anchored to the jawbone 11 by means of suitable fixing devices 12. The fixing devices 12 can be for example resorbable nails. The cavity 10 which is formed between the film 1 and the bone defect site 2 or jawbone 11 can contain bone substitute materials and/or carriers for drugs, growth factors and/or other substances for promoting and protecting healing and bone formation and the provision of a periodontal apparatus around natural teeth to promote bone regeneration. After fitment of the film 1 the gum 13 which has been previously removed or folded back is covered over the film 1 again and suitably sutured. By virtue of the resoption capability of the film and the fixing devices 12 there is no need for any further operation for removing the film 1 and/or the fixing devices 12 again after bone regeneration has occurred.

FIG. 17 shows a bone defect site 2 similar to that shown in FIG. 16, which is covered with a proposed film 1. In this example an implant 14 has been fitted into the jawbone 11 which is still present, the free end of the implant 14 being provided with a screw 15. In this case for easier accessibility to the implant 14 or its screw 15 it can be provided that the film 1 is already provided with a hole which has been previously stamped out and through which the screw 15 can project.

FIG. 18 shows a further example of a bone defect site 2 covered over with a proposed film 1. An implant 14 is already fitted in the jawbone 11, with a post 16 arranged at the free end of the implant 14. The post 16 projects both through the film 1 and also through the gum 13 to facilitate further tooth construction. A support disk 18 is additionally disposed between the post 16 and the film 1. That support disk 18 can provide for closure or sealing-off of the film 1 in that region of the film 1, through which the post 16 penetrates through the film 1 (penetration region). That is important on the one hand to seal off the film 1 with respect to the oral cavity and thus to prevent the occurrence of inflammation. On the other hand it is also possible to provide therewith that the film 1 is resorbed more slowly in precisely that sensitive penetration region and can thus better protect that region. The disk 18 can in that case comprise titanium and project radially beyond the implant 14. The shape of the disk 18 may be for example round or oval. The disk 18 can also be such that it can be cut to shape in order to be able to provide for optimum closure of the penetration region by the film 1 or to stabilize the sensitive defective region around the implant 14, depending on the respective situation of use. In that respect the disk 18 can also be produced in such a way that the film 1 can be clamped and pressed in a groove in the disk 18 as required to achieve stabilization.

FIG. 19 shows a film which is clamped and pressed in a groove in a support disk 18 and FIG. 20 shows the arrangement of that film 1 at a bone defect site 2. In the illustrated example the upper end of the implant 14 (implant head) does not project beyond the jawbone 11 but is disposed beneath the level of the jaw. Depending on how deep the upper end of the implant 14 is disposed in the jawbone 11 or how great the difference in level is between the implant head and the bone level, it is possible to provide a compensation effect by inserts 19 of differing heights so that the film 1 or support disk 18 can be fixed without crater formation. FIG. 21 shows an example of a bone defect site 2 around the natural tooth 17 covered over with a proposed film 1. This example involves the use of the proposed film 1 for covering over a parodontal bone defect site 2. The portions of the jawbone 11 and the tooth 17, which are covered over by the film 1, are shown in broken line.

FIG. 22 shows a sectional view of a jawbone 11 with a bone defect site 2 around a natural tooth 17. To permit bone construction or a production of the periodontal apparatus at the bone defect site 2 a proposed pre-bonded multilayer film 1 is suitably moldingly shaped over the bone defect site 2 and the tooth 17 and anchored to the jawbone 11 by means of suitable fixing devices 12. The fixing devices 12 can be for example resorbable nails. The cavity 10 which is formed between the film 1 and the bone defect site 2 or jawbone 11 can contain bone substitute materials and/or carriers for drugs, growth factors and/or other substances for promoting and protecting healing and bone formation and the production of a periodontal apparatus around natural teeth in order to promote bone regeneration. After fitment of the film 1 the gum 13 which has been previously removed or folded back is covered over the film 1 again and suitably sutured. By virtue of the resorption capability of the film 1 and the fixing devices 12 there is no need for a further operation to remove the film 1 and/or the fixing devices 12 again after bone regeneration has taken place.

1. A pre-bonded multilayer film (1) for covering a bone defect site (2), in particular in the region of a jawbone, wherein the film (1) includes a shaping molding layer (3) for shaping the film (1) to the bone defect site (2) and at least one cover layer (4, 4a, 4b) for covering the bone defect site (2), wherein the molding layer (3) and the at least one cover layer (4, 4a, 4b) are substantially completely resorbable, wherein the at least one cover layer (4) at least partially and preferably substantially completely comprises a bioresorbable collagen material and wherein the molding layer (3) at least partially and preferably substantially completely comprises a bioresorbable polymer material.

2. A pre-bonded multilayer film (1) as set forth in claim 1 wherein the molding layer (3) and the at least one cover layer (4, 4a, 4b) are mechanically and/or chemically connected together.

3. A pre-bonded multilayer film (1) as set forth in claim 1 wherein the molding layer (3) and the at least one cover layer (4, 4a, 4b) are substantially completely resorbable in different periods of time.

4. A pre-bonded multilayer film (1) as set forth in claim 1 wherein the film (1) is substantially completely resorbable overall in a period of time of between about 3 and 12 months, preferably between about 4 and 6 months.

5. A pre-bonded multilayer film (1) as set forth in claim 1 wherein the molding layer (3) is stiffer than the at least one cover layer (4).

6. A pre-bonded multilayer film (1) as set forth in claim 1 wherein the molding layer (3), possibly together with the at least one cover layer (4, 4a, 4b), is adapted to be mechanically and/or thermally and/or chemically deformable.

7. A pre-bonded multilayer film as set forth in claim 1 wherein the bioresorbable collagen material includes type-I-collagen and/or type-III-collagen.

8. A pre-bonded multilayer film (1) as set forth in claim 1 wherein the bioresorbable polymer material includes lactic acid, preferably L-lactic acid, and/or derivatives thereof.

9. A pre-bonded multilayer film (1) as set forth in claim 8 wherein the proportion of lactic acid in the bioresorbable polymer material is at least 70%, preferably between about 80% and 95%, particularly preferably substantially about 82%.

10. A pre-bonded multilayer film (1) as set forth in claim 1 wherein the bioresorbable polymer material includes glycolic acid.

11. A pre-bonded multilayer film (1) as set forth in claim 10 wherein the proportion of glycolic acid in the bioresorbable polymer material is at most 30%, preferably between about 15% and 20%, particularly preferably substantially about 18%.

12. A pre-bonded multilayer film (1) as set forth in claim 1 wherein the molding layer (3) and the at least one cover layer (4) have different surface areas.

13. A pre-bonded multilayer film (1) as set forth in claim 12 wherein the molding layer (3) involves a smaller surface area than the at least one cover layer (4).

14. A pre-bonded multilayer film (1) as set forth in claim 1 wherein the at least one cover layer (4) and/or the molding layer (3) is or are substantially flat throughout.

15. A pre-bonded multilayer film (1) as set forth in claim 1 wherein the molding layer (3) has a shaping structure (5) for shaping molding to the bone defect site (2).

16. A pre-bonded multilayer film (1) as set forth in claim 15 wherein the shaping structure (5) has at least portion-wise a convexly and/or concavely curved edge (6) and/or at least portion-wise a convexly and/or concavely curved shape.

17. A pre-bonded multilayer film (1) as set forth in claim 15 wherein the shaping structure (5) has at least one strut-shaped shaping molding element (7).

18. A pre-bonded multilayer film (1) as set forth in claim 15 wherein the shaping structure (5) is substantially grid-shaped.

19. A pre-bonded multilayer film (1) as set forth in claim 15 wherein the shaping structure (5) is provided by at least one reinforcement of the molding layer (3).

20. A pre-bonded multilayer film as set forth in claim 1 wherein the film (1) has a carrier layer (8) for at least one substance which is arranged or which is to be arranged thereon.
